(19) United States
(12) Patent Application Publication
(10) Pub. No.: US 2021/0244945 A1
(43) Pub. Date: Aug. 12, 2021

(54) ARTIFICIAL VISION SYSTEM

(71) Applicant: Institut De Física D'Altes Energies, Bellaterra (ES)

(72) Inventors: Mokhtar CHMEISSANI RAAD, Bellaterra (ES); Jose Gabriel MACÍAS MONTERO, Bellaterra (ES); Martí VERDAGUER MALLORQUÍ, Bellaterra (ES)

(21) Appl. No.: 17/242,533

(22) Filed: Apr. 28, 2021

Related U.S. Application Data
(63) Continuation of application No. PCT/EP2019/079522, filed on Oct. 29, 2019.

(30) Foreign Application Priority Data
Oct. 30, 2018 (EP) 18382763.3

(51) Int. Cl. A61N 1/05; A61N 1/36; A61N 1/378; H02J 50/30

(52) U.S. Cl. CPC A61N 1/0543; H02J 50/30; A61N 1/3787; A61N 1/36046

(57) ABSTRACT

According to one embodiment a visual prosthesis is provided that includes an integrated circuit having a plurality of pixels and an array of electrodes connected to the pixels and configured to excite neurons in a retina. The prosthesis also includes one or more photovoltaic cells and a photodetector. The integrated circuit is configured to process signals from the photodetector to selectively activate the pixels. The present disclosure further relates to artificial vision systems including such prostheses and methods for inserting the prostheses.

### CROSS-REFERENCE TO RELATED APPLICATIONS

This application relates to and claims the benefit and priority to International Application No. PCT/EP2019/079522, filed Oct. 29, 2019, which claims the benefit and priority to European Application No. EP18382763.3 filed on Oct. 30, 2018.

### FIELD

The present disclosure relates to visual prostheses, and particularly retinal implants. The present disclosure further relates to artificial vision systems comprising retinal implants. And also relates to methods for implanting the retinal implant and to methods for providing artificial vision.

### BACKGROUND

Retinitis pigmentosa (RP) is a genetic disorder of the eyes that causes loss of vision. Symptoms include trouble seeing at night and decreased peripheral vision (side vision).

The underlying mechanism involves the progressive loss of photoreceptor cells in the back of the eye. In severe cases, a person goes blind because the photoreceptor cells are no longer sensitive to light. However, in most cases the neurons (Retina Ganglion Cell) of the retina are still functional.

Such neurons can be stimulated with an electrical signal, and if such neurons are stimulated, the optical nerve transfers such stimulation to the visual cortex.

It is known in the art to use a retinal implant intended to provide electrical stimulation of the retina to induce visual perception in blind individuals and it is indicated for use in patients with severe to profound retinitis pigmentosa. A miniature video camera housed in the patient's glasses captures a scene. The video is sent to a small patient-worn video processing unit where it is processed and transformed into instructions that are sent back to the glasses via a cable. These instructions are transmitted wirelessly to an antenna in the retinal implant. The signals are then sent to the electrode array, which emits small pulses of electricity. These pulses stimulate the retina's remaining cells, which transmit the visual information along the optic nerve to the brain, creating the perception of patterns of light. Patients learn to interpret these visual patterns with their retinal implant.

The electrical signals are generated outside the sclera and sent via a cable to inside the eye. Each electrode generates an electric signal independently to excite the neurons underneath the retina's surface.

In one known implant, the electrode array comprises 60 electrodes. Even though patients may recognize very basic shapes thanks to the 60 electrodes, it is clear that a patient may benefit from an increase in electrodes, and from an increased density of electrodes (i.e. electrodes per square area). More neurons or areas might be excited and thus more detailed information on shapes might be transmitted to the optical nerve, potentially leading to improved vision.

However, an increase in the number of electrodes means an increase in power consumption and also in the size of data transfer to configure the matrix of electrodes for each frame image. It is evident that to increase the number of electrodes and still provide them with sufficient power is not a trivial matter.

Moreover, a significant increase of electrodes with good signal quality is also hard to realize due to the increase in electrical wires necessary.

The present disclosure provides examples of systems and methods that at least partially resolve some of the aforementioned disadvantages.

### SUMMARY

In a first aspect, a visual prosthesis is provided. The visual prosthesis comprises an integrated circuit comprising a plurality of pixels, and an array of electrodes attached to the pixels and configured to excite neurons in a retina. The prosthesis furthermore comprises one or more photovoltaic cells, and a photodetector, and the integrated circuit is configured to process signals from the photodetector to selectively activate the pixels.

According to this aspect, the retinal implant can be provided with many more individual electrodes or pixels which can increase the quality of vision for patients, as more detailed shapes may be excited. The photovoltaic (PV) cells can be powered by incoming lights. Incoming light flashes or pulses may be registered by the photodetector and the integrated circuit can analyse the received pulses and transform into a selective activation of the pixels. Incoming light can thus both be used to power the implant and to transmit information that might be obtained from a camera.

Moreover, signal quality can be maintained even if the number of pixels increases as the electrodes may be directly attached to the pixels of the integrated circuit. Cross-talk between wires can be effectively avoided.

In the present disclosure, a visual prosthesis may be regarded as any visual device intended to (partly) restore functional vision in those suffering from partial or total blindness.

In particular, the visual prosthesis may be a retinal implant. In the present disclosure, the term retinal implant is meant to cover epiretinal implants (i.e. implants which in use are positioned on the retina), subretinal implants (which are to be implanted behind the retina), and suprachoroidal implants (which are to be implanted between the choroid and the sclera).

In some examples, the visual prosthesis (retinal implant) may comprise a capacitor, and optionally the capacitor is a graphene layer. The capacitor allows the storage of electrical energy derived from the PV cells.

In some examples, the integrated circuit may be an ASIC, i.e. a chip specifically developed for the specific use as herein described.

In some examples, the photodetector may be a PIN diode.

In some examples, the visual prosthesis may be configured to assume an unfolded state and a folded state. The provision of PV cells in the visual prosthesis means that the size of the implant is increased. This could lead to a more problematic process of implanting it. This can be at least partially resolved when the visual prosthesis can be implanted in a folded state, and once in an appropriate position within the eye, the implant can unfold.

One way of implementing the ability to fold or to deploy is by using shape memory material. Shape memory material may be activated in a variety of ways, e.g. if the material is exposed to a change in temperature.

In a further aspect, an artificial vision system is provided. The artificial vision system comprises a visual prosthesis, and particularly a retinal implant according to any of the examples herein described, and further comprises a camera, an image processor for analysing a video signal from the camera to generate a data pattern, and a light source for emitting light according to the generated data pattern.

In this aspect, a functional kit is provided for operating the visual prosthesis.

In some examples, the light source may comprise a LED. Specifically, the LED may be configured to emit light with a wavelength in a range of 700 nm or more, particularly 750 nm or more, more particularly 800 nm or more. At these wavelengths, the light can be transmitted through the patient's eye (including lens, cornea and vitreous humor) without significant losses. A retinal implant may thus be used. In particular, the implant may be an epi-retinal implant, i.e. positioned within the retina. Moreover, the PV cells, if selected properly may function at high efficiency at these wavelengths.

In other examples, the implant may be a subretinal implant (i.e. behind the retina), or a suprachordial implant (between the choroid and sclera).

In some examples, the camera, the image processor and the light source are mounted on a support, and in particular on glasses. A patient may wear the glasses and the camera is therewith positioned close to the eyes and the images recorded by the camera are recorded from an angle of vision of the natural eyes.

In yet a further aspect, a method for implanting a retinal implant is provided. The method comprises: providing a retinal implant according to any of the examples herein disclosed, and making an incision in a sclera. After making the incision in the sclera, the retinal implant may be inserted through the incision, when the retinal implant is in a folded state. After insertion, the retinal implant may be unfolded.

In some examples, the retinal implant may be provided in a sheath, and unfolding the retinal implant comprises removing the sheath.

In yet a further aspect, a method for providing artificial vision for a patient is provided. The method comprises recording an image, processing the recorded image to generate a pixelated image, and producing light pulses transmitting the generated pixelated image to a visual prosthesis. The visual prosthesis may be a retinal implant, and more specifically an epiretinal implant.

In accordance with this aspect data transmission using light is used for communication between a visual prosthesis and an image recording system (e.g. a camera or video camera). The light used for data transmission may be in the visual light spectrum, but preferably will be in the infrared range.

### BRIEF DESCRIPTION OF THE DRAWINGS

Non-limiting examples of the present disclosure will be described in the following, with reference to the appended drawings, in which:

FIG. 1 schematically illustrates a prior art retinal implant;

FIGS. 2A-2D schematically illustrate a first example of a retina implant and an artificial vision system including such a retinal implant;

FIGS. 3A and 3B schematically illustrate an example of a retinal implant;

FIGS. 4A-4G schematically illustrate a method of implanting according to an example; and

FIG. 5 schematically illustrates a further example of a retina implant and an artificial vision system including such a retinal implant.

### DETAILED DESCRIPTION

In these figures the same reference signs have been used to designate the same elements.

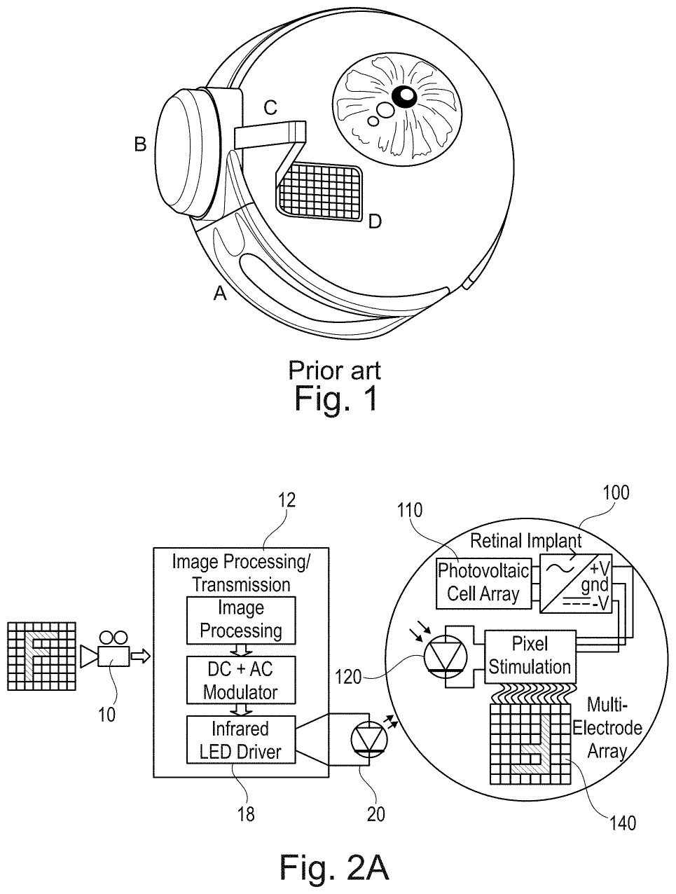

FIG. 1 schematically illustrates a prior art retinal implant. The implant comprises an antenna A which receives signals from a further antenna which may be integrated in glasses worn by a user. The signals sent to antenna A may be the result of image processing of a video integrated in the glasses.

Reference sign B refers to an electronic unit outside the eye. Individual electrical cables C lead from the electronics unit to electrode array D.

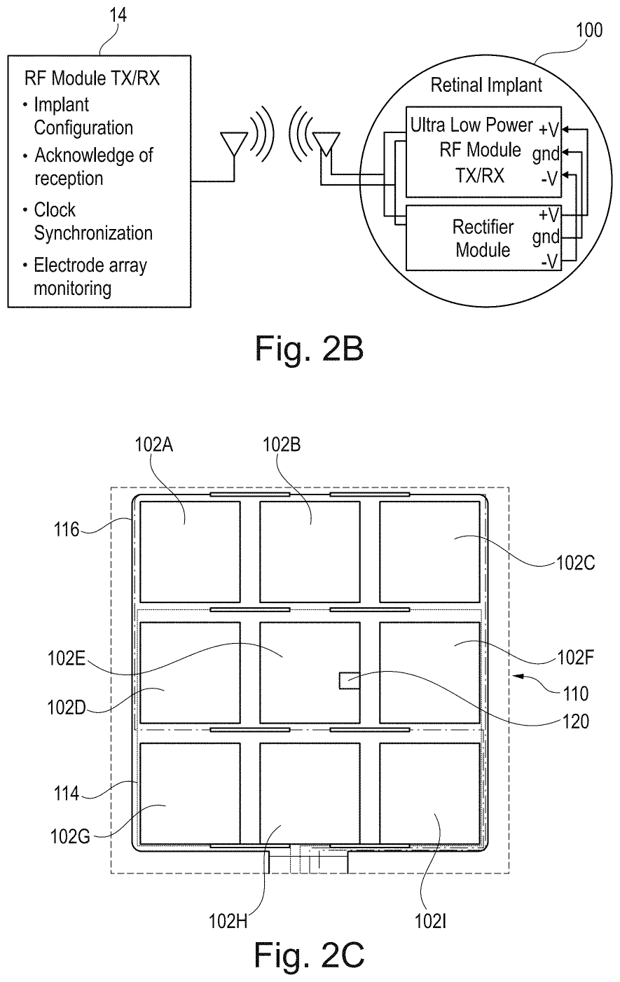

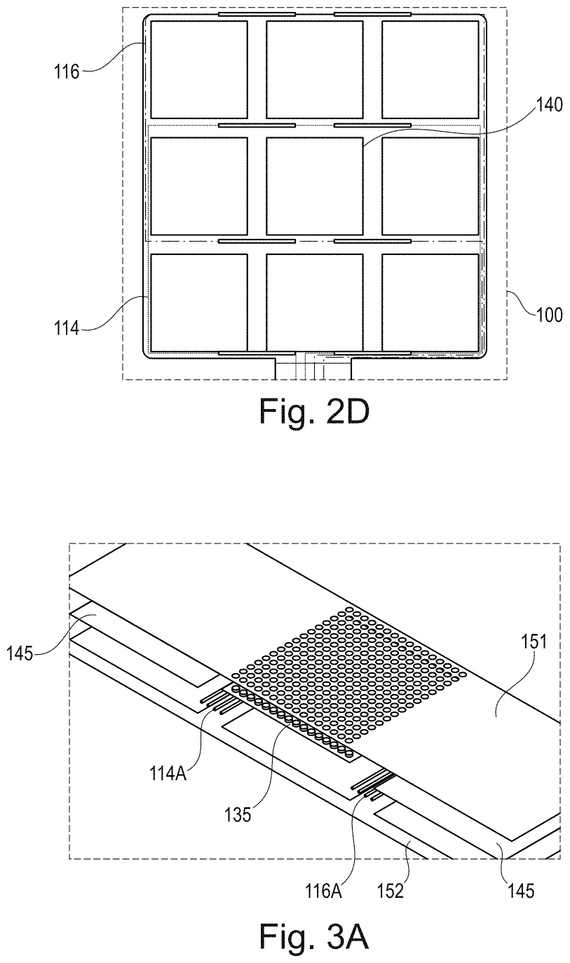

FIGS. 2A-2D schematically illustrate a first example of a retina implant and an artificial vision system including such a retinal implant. The artificial vision system according to this example comprises an implant 100. The implant 100 may be an epi-retinal implant.

The system according to this example furthermore comprises a camera 10. The camera 10 may be a digital camera, and specifically a digital camera integrated in glasses that may be worn by a user.

Recordings from camera 10 may be sent in real-time to an image processor 12 for analysing a video signal from the camera to generate a data pattern. The data pattern may be generated by infrared LED driver 18. The LED driver 18 in the example drives the flashing of LED 20. The flashing of LED may have a frequency of e.g. 1 MHz-1 GHz, specifically 10-500 MHz. Both the LED driver 18 and LED 20 may be integrated in the same glasses worn by the user.

The light flashes from LED 20 may be received by a photoreceptor 120 of implant 100. The photoreceptor 120 may be a PIN diode in some examples. Implant 100 includes an integrated circuit, i.e. a chip. Signals received by the photoreceptor 120 may be transformed into excitation signals for the electrodes 140.

In particular, the photoreceptor and circuit may be programmed such that an incoming light flash causes an electric pulse and the electrical pulse is compared to a threshold. If the pulse is above the predetermined thresholds, a “1” is generated. If the pulse is below the threshold (e.g. absent), then a “0” is generated. By sending light pulses at very high frequencies, information is transmitted about the excitation of individual electrodes in electrode array 140. I.e. based on the “1”s and “0”s received, it may be determined which of the electrodes have to be excited.

The implant 100 may be powered by a photovoltaic cell array 110 connected to a suitable converter.

Light is thus used herein both for transmission of information and for transmission of power. There is thus no need for a sizable battery in the implant.

FIG. 2B schematically illustrates a high level communication between the glasses 14 (or other support) including the camera, image processor and LED and implant 100. The glasses 14 in this example comprise a first radiofrequency transceiver (rf transmitter/receiver) and the implant 100 comprises a second rf transceiver.

As illustrated in FIG. 2B, basic functionalities may be communicated through the rf transmitter, including synchronization of the glasses 14 (and thus the video signal and image processing) with the implant (and thus the excitation of individual electrodes), so that the excitation of electrodes is in accordance with the camera recordings. Other basic communications may include e.g. a fault communication, and an acknowledgement of receipt.

FIG. 2C schematically illustrates that in this example, the PV array includes 9 PV cells 102A-102I. In an illustrative example, the PV cells may be PERT cells which have a high efficiency. Each of the PV cells may have an area of 15-40 mm2, particularly approximately 25 mm2. In the depicted example, 9 PV cells of each 5 by 5 mm may be used. The chip with the attached electrodes may also have a size of approximately 5 by 5 mm in this example. The number of pixels and electrodes may be e.g. 15 by 22, 20 by 30, or 25 by 33 or more. As compared to the prior art illustrated in FIG. 1, the number of electrodes may thus be multiplied by 10 or more. Note that in these examples an approximate ratio of height to width of 2:3 is maintained. Variations with respect to this ratio are possible.

In an example, PERT PV cells may be used with a thickness of approximately 100 microns, e.g. in the range of 50-300 microns.

It should be clear that the number of PV cells, the type and the area occupied by the cells may be adjusted in accordance with energy needs. Also another shape may be selected for the PV array.

If the frequency of flashing of the LED is high enough, for example in the aforementioned frequency ranges, the light will be seen as substantially continuous by the PV cells, so that the performance is not negatively affected.

In the central PV cell 102 E, photoreceptor 120 may be integrated.

In an example, the LED 20 may be configured to emit light with a wavelength in a range of 700 nm or more, particularly 750 nm or more, more particularly 800 nm or more. At these wavelengths, the PV cells have a high efficiency, and moreover, the transmission through the human eye (including cornea, lens, vitreous humor) is high as well, for example 85% or more or 90% or more.

In the example of FIG. 2D, the retinal implant 100 is configured to assume an unfolded state and a folded state. The implant 100 includes a first frame of shape memory material 114 which when activated forces the implant to the folded shape, and a second frame 116 of shape memory material which when activated forces the implant to the unfolded shape.

In this example, the shape memory material may be thermally activated and particularly may include shape memory metal. The areas around the junctions between the columns of the PV cells may have a memory shape so that the junction is straight or folded. By sending an electrical current through the metal frame 114 or 116, one of the frames may be heated so that the frame has a tendency to assume its memory shape. If the frame 114 is activated, the junctions between the three columns will fold, such that the implant as a whole assumes the folded shape. If the frame 116 is activated, the junctions between the columns of cells will straighten out, such that the implant will assume an unfolded state.

In examples, the temperature of transition from one shape to another shape may be well above 37° C., i.e. normal body temperature. If the temperature is chosen close to the average body temperature, a variation of temperature due to e.g. a fever may provoke a change of shape of the implant.

It should be clear that in another example, the tendencies of the shape memory frames may be exchanged. It should also be clear that other shape memory materials and activation methods may be used.

FIG. 2D shows the same PV cell array viewed from the opposite side. The bottom sides of the PV cells may be seen. At the bottom of the PV cells, a reflective layer may be provided, e.g. an aluminized Mylar™ layer, to reflect back any possible incident photon that has passed through the PV cells.

Underneath the central PV cell, the chip with electrode array 140 may be arranged.

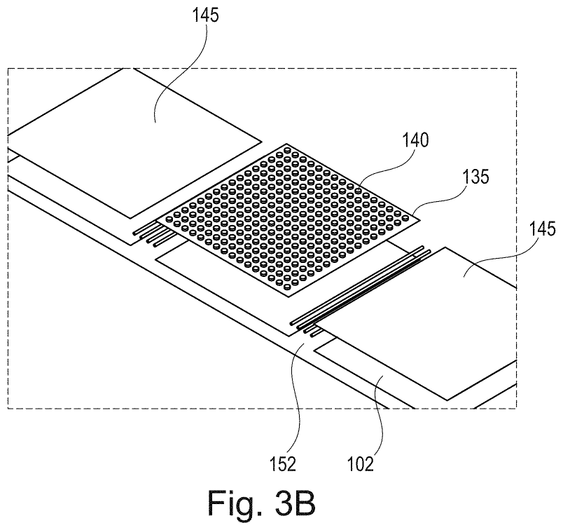

FIGS. 3A and 3B schematically illustrates an example of a retinal implant. In FIG. 3, three PV cells are shown, whereas in FIG. 2 nine PV cells were shown. The illustration in FIG. 3 may be understood as a separate example of the retinal implant, but it should be clear that the build-up shown in FIG. 3 may also be regarded as illustrating only a portion of the implant shown in FIG. 2, i.e. the illustration in FIG. 3 should not be understood as limiting in size, number of PV cells etc.

The retinal implant comprises a base 152 and the PV cells may be mounted on base 152. The base 152 may be e.g. a polyamide.

The retinal implant further comprises a capacitor 145 to store energy generated by the PV cells. The capacitor 145 may be a graphene layer, and in particular an array of graphene areas 145 connected to each of the individual PV cells. If the same configuration is assumed as in FIG. 2, there may be nine areas of approximately 5 mm by 5 mm of graphene. The thickness of the graphene layer may be e.g. between 50 and 500 microns. Assuming a thickness of approximately 100 microns, and assuming an estimated capacitance of at least 200 F/g, one may expect to store 18 Joules. If the capacitance of graphene is improved, or the area or thickness of graphene are increased, more energy may evidently be stored.

However, on top of one of the PV cells, an ASIC 135 may be provided. In the configuration of FIG. 2, the ASIC may be provided on the central PV cell. An array of electrodes 140 may be attached to ASIC 135, in particular the electrodes may be mounted directly, i.e. deposited on the ASIC 135.

In the ASIC, a plurality of pixels may be defined which can be individually excited in accordance with the instructions received from the LED.

In the example of FIG. 3A, the implant also comprises a top layer 151, and the top layer 151 may be made from polyamide. The top and bottom layers may serve the purpose of reducing the exposure of components of the implant to the surroundings.

In order to allow the electrode to stimulate cells in the eye, the top layer 151 may comprise a plurality of holes corresponding to the electrodes.

Reference signs 114A and 116A illustrate stretches of shape memory material, which may in particular be located between PV cells, and particularly between rows of PV cells, such that the columns may be folded as illustrated in the example of FIG. 2. The shape memory material may be metallic, and in the illustrated example, the shape memory material may be e.g. Nitinol.

FIG. 3B illustrates the same implant (or part of implant) as shown in FIG. 3A. For the purposes of enhancing the understanding, the top layer 150 has been removed in this figure. The ASIC 135 with electrodes 140 may easily be recognized, as well as graphene areas 145 placed on PV cells 102.

With the build-up of FIGS. 2 and 3 for a foldable implant, the maximum thickness for the implant when folded is preferably below 1 mm. In an example, the thickness of the PV cells is chosen at 100 microns, and the same thickness is chosen for the graphene layer.

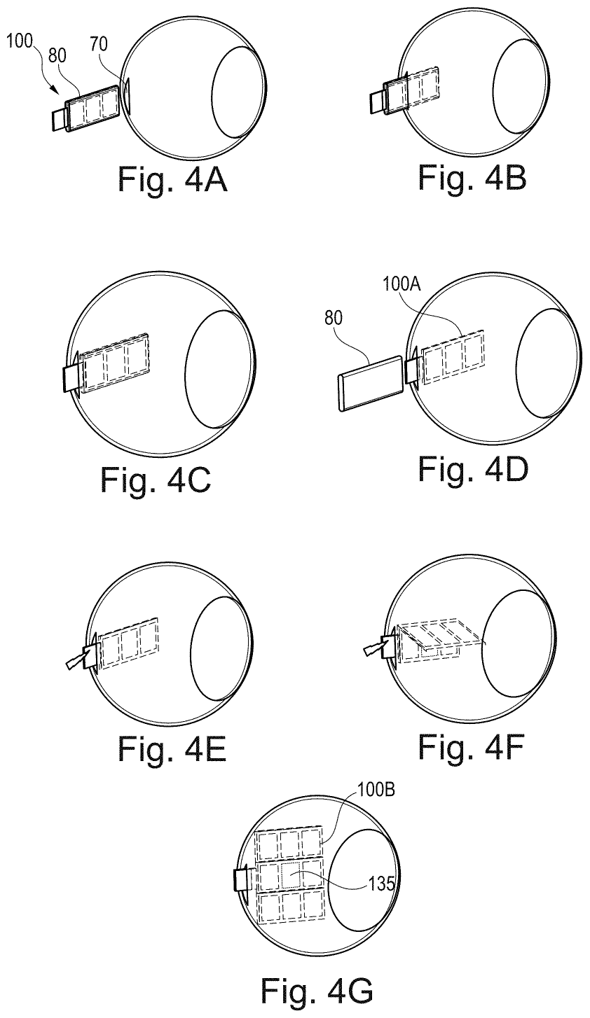

FIGS. 4A-4G schematically illustrates a method of implanting according to an example. The implant illustrated in FIG. 4 substantially corresponds to the implant illustrated particularly in FIG. 2, but the same method or similar methods may be used in different implants as well.

In FIG. 4A, an incision 70 may be made in a patient's eye. The implant 100 in this example is provided in a sheath 80. The implant, as will become clear from the following figures, is in a folded state. A small incision is therefore sufficient for the introduction of the implant 100.

FIGS. 4B and 4C schematically illustrate how implant 100 arranged within sheath 80 is inserted into the eye. In FIG. 4D, it is shown how sheath 80 may be removed, while the implant is still in the folded state 100A. In FIG. 4E, an electrical current may be sent through the nitinol wires illustrated in the previous figures. By sending electrical current through the nitinol wires, these wires may heat up and thus assume their remembered shape. As was explained with reference to FIG. 2, the remembered shape of a frame may be the unfolded or stretched out shape of the wires.

As a result, in FIG. 4F, implant starts to unfold, to assume in FIG. 4G the unfolded state 100B. If and when necessary, the implant may be removed by sending electrical current through the other shape memory frame so increase the heat and stimulate that frame to assume its folded shape. Once the implant has assumed its folded state 100A, the implant may be removed. In FIG. 4G, ASIC 135 (located below the PV cell) is indicated for better reference.

In an alternative implant, and an alternative method for inserting the implant, the implant may be designed to assume its unfolded state without any external stimulation. I.e. merely by removing the sheath 80, the implant will assume the unfolded shape.

It will be clear that the implant, including PV cells, photoreceptor, integrated circuit with pixels attached to electrodes is entirely placed within the eye. The previously illustrated wires for the prior art leading from outside the eye to inside the eye are thus avoided, while energy supply can be ensured.

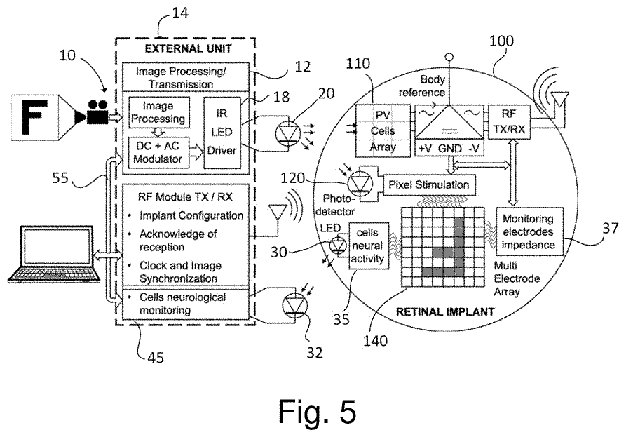

FIG. 5 schematically illustrates a further example of a retina implant 100 and an artificial vision system including such a retinal implant. The artificial vision system in this example includes a camera 10, an image processor 12 for analysing a video signal from the camera to generate a data pattern, and a light source 20 for emitting light according to the generated data pattern. The camera 10 and image processor may be arranged on an external unit or support 14, e.g. glasses worn by a patient.

The functioning of the implant 100 and artificial vision system is largely comparable to the functioning of the example in FIG. 2. The data pattern derived from the video signal after image processing is transmitted to the implant 100. The LED driver 18 in the example drives the flashing of LED 20. Data transmission in this example occurs through light. Photodetector diode 120 receives the light and the data pattern is analysed to stimulate the pixels as in the example of FIG. 2.

The light flashes from LED 20 may be received by a photoreceptor 120 of implant 100. The photoreceptor 120 may be a PIN diode in some examples. Implant 100 includes an integrated circuit, i.e. a chip. Signals received by the photoreceptor 120 may be transformed into excitation signals for the electrodes 140.

Simultaneously, the light is received by PV cells array 110 to provide power for the functioning of the implant 100.

However, in this specific example, a further functionality is included. If and when a pixel is stimulated, the neurons situated behind and in the immediate vicinity are stimulated. There are however different types of neurons, and upon implanting the retinal implant, the precise disposition of pixels with respect to neurons is not known.

When a pixel is stimulated, not only neurons underlying the specific pixel receive an electric charge, but also neurons underlying neighbouring pixels notice the excitation. By measuring the response at electrodes for pixels neighbouring the pixel that is actually stimulated, neural activity may be derived (block 35) including information on the quantity and types of neurons underlying various pixels.

It may be derived for example, that there is no neural activity under some of the pixels. Exciting these pixels is thus not useful in this case. It may be better to activate a neighbouring pixel instead to approximate the image recorded by the camera. It may also be derived that e.g. neurons underlying some of the pixels are more active than neurons underlying other pixels. If such information can be taken into account in the excitation of the pixels, the visualization of the image recorded by camera 10 may be improved for patients. In some examples, information regarding neural activity may be correlated with specific pixels. In some examples, the information regarding neural activity in relation with pixels may be transmitted to the image processor 12.

In this particular example, the data may be transmitted through light as well. The retinal implant in this example includes a light source 30, e.g. a LED. The light signal emitted from LED 30 may be modulated to transmit data. The light may be received at photodetector 32. The signals received by photodetectors 32 may be analysed (block 45) and the information regarding neural activity in relation with individual pixels/electrodes may be stored and/or fed back (block 55) to the image processor 12. In subsequent image processing to determine appropriate stimulation of pixels/electrodes, the knowledge on neural activity derived from previous stimulations may be used to improve the visual perception of the patient.

The information feedback regarding neural activity (block 35) and transmission through LED 30 may be carried out at a lower frequency than the transmission of light from LED 20. In some examples, information may be gathered during some time before transmitting the information.

In the example of FIG. 5, (as in the example of FIG. 2) a high level communication between the glasses (or other support) including the camera, image processor and LED and implant 100. The support 14 in this example comprise a first radiofrequency transceiver (rf transmitter/receiver) and the implant 100 comprises a second rf transceiver.

Basic functionalities may be communicated through the rf transmitter, including synchronization of the glasses (and thus the video signal and image processing) with the implant (and thus the excitation of individual electrodes), so that the excitation of electrodes is in accordance with the camera recordings. Other basic communications may include e.g. a fault communication, and an acknowledgement of receipt. In this particular example, the synchronization also serves to correlate the electrode response that is measured with specific excitations of pixels.

The second rf transmitter may also communicate the electrode impedance of the pixel array and individual pixels. Excitation may be adapted as a function of the measured or determined impedance.

In a further example, the transmission of information regarding neural activity might occur through the second rf transceiver to first rf transceiver on support 60.

For reasons of completeness, various aspects of the present disclosure are set out in the following numbered clauses:

Clause 1. A visual prosthesis comprising an integrated circuit comprising a plurality of pixels, an array of electrodes connected to the pixels and configured to excite neurons in a retina, one or more PV cells, and a photodetector, wherein the integrated circuit is configured to process signals from the photodetector to selectively activate the pixels.

Clause 2. A visual prosthesis according to Clause 1, wherein each of the electrodes is mounted on one of the pixels.

Clause 3. A visual prosthesis according to Clause 1 or 2, further comprising a capacitor.

Clause 4. A visual prosthesis according to Clause 3, wherein the capacitor is a graphene layer.

Clause 5. A visual prosthesis according to any of Clauses 1-4, further comprising a base layer, the PV cells being mounted on the base layer.

Clause 6. A retinal implant according to Clause 5, wherein the base layer is made from polyamide.

Clause 7. A visual prosthesis according to Clause 5 or 6, further comprising a top layer, and wherein the top layer is optionally made from polyamide.

Clause 8. A visual prosthesis according to Clause 7, wherein the top layer comprises a plurality of holes corresponding to the electrodes.

Clause 9. A visual prosthesis according to any of Clauses 1-8, wherein the integrated circuit is an ASIC.

Clause 10. A visual prosthesis according to any of Clauses 1-9, wherein the photodetector is a PIN diode.

Clause 11. A visual prosthesis according to any of Clauses 1-10, comprising three columns of PV cells.

Clause 12. A visual prosthesis according to Clause 11, wherein each of the columns comprises three PV cells.

Clause 13. A visual prosthesis according to any of Clauses 1-12, wherein the retinal implant is configured to assume an unfolded state and a folded state.

Clause 14. A visual prosthesis according to Clause 13, comprising shape memory material.

Clause 15. A visual prosthesis according to Clause 14, comprising a first frame of shape memory material which when activated forces the implant to the folded shape, and a second frame of shape memory material which when activated forces the implant to the unfolded shape.

Clause 16. A visual prosthesis according to Clause 14 or 15, wherein the shape memory material is thermally activated.

Clause 17. A visual prosthesis according to any of Clauses 1-16, wherein the PV cells are PERT cells.

Clause 18. A visual prosthesis according to any of clauses 1-17, further comprising a system for analysing neural activity in response to activation of the pixels.

Clause 19. A visual prosthesis according to clause 18, wherein the system for analysing neural activity is configured to determine electrical excitation at pixels neighbouring an activated pixel.

Clause 20. A visual prosthesis according to clause 18 or 19, further comprising a system for transmitting neural activity.

Clause 21. A visual prosthesis according to clause 20, wherein the system for transmitting neural activity includes a light source for emitting information relating to the neural activity.

Clause 22. A visual prosthesis according to clause 21, wherein the light source is a LED.

Clause 23. A visual prosthesis according to clause 21 or 22, wherein light transmission of the light source is modulated to transmit information.

Clause 24. A visual prosthesis according to any of clauses 1-23, further comprising a system for monitoring impedance of the array of electrodes.

Clause 25. An artificial vision system comprising:

the visual prosthesis according to any of Clauses 1-24, a camera,
an image processor for analysing a video signal from the camera to generate a data pattern, and a light source for emitting light according to the generated data pattern.

Clause 26. An artificial vision system according to Clause 25 wherein the light source comprises a LED.

Clause 27. An artificial vision system wherein the LED is configured to emit light with a wavelength in a range of 700 nm or more, particularly 750 nm or more, more particularly 800 nm or more.

Clause 28. An artificial vision system according to any of Clauses 25-27, wherein the camera, the image processor and the light source are mounted on a support, in particular on glasses.

Clause 29. An artificial vision system according to Clause 28, wherein the glasses comprise a first rf transceiver, and the visual prosthesis comprises a second rf transceiver.

Clause 30. An artificial vision system according to Clause 29, wherein the first rf transceiver and the second rf transceiver are configured to communicate to synchronize a clock of the glasses with a clock of in the integrated circuit.

Clause 31. An artificial vision system according to any of clauses 28-30, wherein the support further comprises a photodetector for receiving light emitted from the visual prosthesis.

Clause 32. A method for implanting a retinal implant comprising:

providing a visual prosthesis according to any of Clauses 13-16;
making an incision in a sclera; inserting the visual prosthesis through the incision, wherein the retinal implant is in a folded state, and unfolding the visual prosthesis.

Clause 33. The method according to Clause 32, wherein the visual prosthesis is provided in a sheath, and unfolding the visual prosthesis comprises removing the sheath.

Clause 34. A method for providing artificial vision for a patient comprising: recording an image, processing the recorded image to generate a pixelated image, producing light pulses transmitting the generated pixelated image to a visual prosthesis, specifically a retinal implant, and more specifically an epiretinal implant.

Clause 35. A method according to Clause 34, wherein images are recorded continuously, and the recorded images are processes in real-time to generate consecutive pixelated images, and light pulses are produced continuously to transmit the consecutive pixelated images to the retinal implant.

Clause 36. A method according to Clause 34 or 35, wherein light pulses are produced at a frequency between 1 MHz and 1 GHz.

Clause 37. A method according to any of Clauses 34-36, further comprising: a photoreceptor receiving the light pulses and analysing the received light flashes to selectively excites electrodes in an array of electrodes.

Clause 38. A method according to any of Clauses 34-37, further comprising: one or more photovoltaic cells receiving the light pulses to generate electricity, and using the generated electricity for supplying the retinal implant.

Clause 39. A method according to any of clauses 34-38, further comprising deriving information relating to neural activity in correlation with the pixels.

Clause 40. A method according to clause 39, wherein the deriving information relating to neural activity comprises measuring an electric response at pixels in a vicinity of a pixel being activated, and specifically at pixels neighbouring the pixel being activated.

Clause 41. A method according to clause 40, further comprising transmitting the information relating to neural activity, optionally transmitting the information relating to neural activity to a support carrying a camera for the recording of the image.

Clause 42. A method according to clause 41, wherein the support is a pair of glasses.

Clause 43. A method according to clause 40 or 41, wherein the transmitting the information relating to neural activity comprises producing light pulses transmitting the information to a photoreceptor,

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

What is claimed is:

1. A visual prosthesis comprising:
an integrated circuit comprising a plurality of pixels;
a plurality of electrodes, each of the plurality of electrodes being electrically coupled to one of the plurality of pixels and configured to excite neurons in a retina when the pixel is activated;
a photodetector that is configured to receive light pulses and to generate output signals in response to the received light pulses, the integrated circuit being configured to process the output signals from the photodetector to selectively activate the plurality of pixels; and
one or more photovoltaic cells electrically coupled to the integrated circuit and being configured to produce electricity to power the integrated circuit.

2. The visual prosthesis according to claim 1, wherein each of the plurality of electrodes is mounted on the one of the plurality of pixels.

3. The visual prosthesis according to claim 1, further comprising a capacitor electrically coupled to the one or more photovoltaic cells and being configured to store electrical power produced by the one or more photovoltaic cells, the capacitor being a graphene layer.

4. The visual prosthesis according to claim 1, further comprising a base layer, the one or more photovoltaic cells being mounted on the base layer.

5. The visual prosthesis according to claim 4, further comprising a top layer overlying the plurality of electrodes, the top layer including a through hole for each of the plurality of electrodes.

6. The visual prosthesis according to claim 1, wherein the photodetector is a PIN diode.

7. The visual prosthesis according to claim 1, wherein the visual prosthesis is configured to assume an unfolded state and a folded state.

8. The visual prosthesis according to claim 7, comprising a first frame made of a first shape memory material that when activated causes the visual implant to assume the folded shape, and a second frame made of a second shape memory material that when activated causes the visual prosthesis to assume the unfolded shape.

9. An artificial vision system comprising:
a camera configured to produce an output video signal;
an image processor configured to receive the output video signal and to generate a data pattern based on the received output video signal;
a light source configured to emit light according to the generated data pattern; and
a visual prosthesis comprising:
an integrated circuit comprising a plurality of pixels,
a plurality of electrodes, each of the plurality of electrodes being connected to at least one of the pixels and configured to excite neurons in a retina when the at least one pixel is activated,
a photodetector that is configured to receive light pulses and to generate output signals in response to the received light emitted by the light source, the integrated circuit being configured to process the output signals from the photodetector to selectively activate the plurality of pixels; and
one or more photovoltaic cells electrically coupled to the integrated circuit and being configured to produce electricity to power the integrated circuit.

10. The artificial vision system according to claim 9, wherein the light source comprises a light emitting diode.

11. The artificial vision system according to claim 10, wherein the light emitting diode is configured to emit light with a wavelength of 700 nm or more.

12. The artificial vision system according to claim 10, wherein the light emitting diode is configured to emit light pulses at a frequency between 1 MHz and 1 GHz.

13. The artificial vision system according to claim 9, wherein the camera, the image processor and the light source are mounted on a common support.

14. The artificial vision system according to claim 13, wherein the support is a pair of glasses.

15. The artificial vision system according to claim 14, wherein the glasses comprise a first rf transceiver, and the visual prosthesis comprises a second rf transceiver.

16. The artificial vision system according to claim 15, wherein the first rf transceiver and the second rf transceiver are configured to communicate with one another to synchronize a clock of the glasses with a clock of the integrated circuit.

17. A method for providing artificial vision for a patient comprising:
recording an image;
processing the recorded image to generate a pixelated image;
producing light pulses transmitting the generated pixelated image to a retinal implant.

18. The method according to claim 17, wherein the image is recorded continuously, the recorded image being processed in real-time to generate consecutive pixelated images, the light pulses being produced continuously to transmit the consecutive pixelated images to the retinal implant.

19. The method according to claim 18, wherein light pulses are produced at a frequency between 1 MHz and 1 GHz.

20. The method according to claim 17, further comprising a photoreceptor configured to receive the light pulses and to analyse the received light pulses to selectively excite electrodes in an array of electrodes.
